(19) United States
(12) Patent Application Publication
(10) Pub. No.: US 2025/0041177 A1
(43) Pub. Date: Feb. 6, 2025

(54) PREPARATION OF HYALURONIC ACID CBD CONJUGATES

(71) Applicants: Ariel Scientific Innovations Ltd., Ariel (IL); Biosoft Australia Pty Ltd, Belrose (AU)

(72) Inventors: Gary GELLERMAN, Rishon-LeZion (IL); Andrii BAZYLEVICH, Ariel (IL)

(73) Assignees: Ariel Scientific Innovations Ltd., Ariel (IL); Biosoft Australia Pty Ltd, Belrose (AU)

(21) Appl. No.: 18/926,365

(22) Filed: Oct. 25, 2024

Related U.S. Application Data
(63) Continuation of application No. 17/591,739, filed on Feb. 3, 2022, now abandoned.
(60) Provisional application No. 63/145,598, filed on Feb. 4, 2021.

(51) Int. Cl. A61K 8/34; A61K 8/73; A61Q 19/02

(52) U.S. Cl. CPC A61K 8/347; A61K 8/735; A61Q 19/02; A61K 2800/91

(57) ABSTRACT

A molecular structure comprising a single-stranded hyaluronic acid moiety, and a plurality of releasable cannabidiol moieties attached thereto is provided herein, as well as uses and methods of treating a skin conditions using the same.

### RELATED APPLICATIONS

This application is a continuation of U.S. patent application Ser. No. 17/591,739, filed on Feb. 3, 2022 which claims the benefit of priority under 35 USC § 119 (e) of U.S. Provisional Patent Application No. 63/145,598 filed on Feb. 4, 2021, the contents of which are incorporated herein by reference in their entirety.

### FIELD AND BACKGROUND OF THE INVENTION

The present invention, in some embodiments thereof, relates to cosmetics, and more particularly, but not exclusively, to a molecular structure based on multiple and releasable cannabidiol (CBD) moieties carried by hyaluronic acid (HA) as drug-delivery vehicle, and to uses thereof.

Hyaluronic acid (HA), a naturally-occurring glycosaminoglycan (GAG), plays a key role in healing various skin conditions. HA has a range of naturally occurring molecular sizes from 100 to 10,000,000 Da. HA is implicated in water homeostasis of tissues, in the regulation of permeability of other substances by steric exclusion phenomena, and in the lubrication of joints. HA also binds specifically to proteins in the extracellular matrix, on the cell surface, and within the cells cytosol, thereby having a role in cartilage matrix stabilization, cell motility, growth factor action, morphogenesis and embryonic development and inflammation. Unmodified HA has many important application in drug delivery and surgery. For example, it is used as an adjuvant for ophthalmic drug delivery. In addition, HA has important application in the fields of visco-surgery, visco-supplementation and wound healing. HA is also a building-block for biocompatible and biodegradables polymers with application in drug delivery, tissue engineering and visco-supplementation.

Cannabidiol (CBD) is a phytocannabinoid discovered in 1940, and is one of more than a hundred identified cannabinoids in cannabis plants and accounts for up to 40% of the plant's extract. As of 2019, clinical research on CBD included studies related to anxiety, cognition, movement disorders, pain, antimicrobial and antifungal activity, as well as other medical and cosmetic conditions.

Cannabidiol can be taken in multiple ways, including by inhalation of cannabis smoke or vapor, as an aerosol spray into the cheek, by mouth and by transdermal and subcutaneous modes of administration. It may be supplied as CBD oil containing only CBD as the active ingredient (excluding tetrahydrocannabinol [THC] or terpenes), CBD-dominant hemp extract oil, capsules, dried cannabis, or prescription liquid solution. CBD does not have the same psychoactivity as THC, and may change the effects of THC on the body if both are present.

In the United States, the cannabidiol drug Epidiolex was approved by the Food and Drug Administration in 2018 for the treatment of two epilepsy disorders. Since cannabis is a Schedule I controlled substance in the United States, other CBD formulations remain illegal under federal law to prescribe for medical use or to use as an ingredient in dietary supplements or other foods.

U.S. Pat. No. 8,293,786 describes cannabidiol prodrugs, methods of making cannabidiol prodrugs, formulations comprising cannabidiol prodrugs and methods of using cannabidiols, including transdermal or topical administration of a cannabidiol prodrug for treating and preventing diseases and/or disorders.

U.S. Patent Application Publication No. 2015024599 discloses an anti-aging composition for dermal application comprising cannabidiol hemp oil, chuanxiong extract, mondo grass extract, Chinese foxglove extract, female and Panax ginseng extract, dragon's blood resin, lilyturf root, and jojoba oil. The document further discloses other active ingredients that may include licorice root, Astragalus membranceus, tree peony, mulberry bark extraction, longan fruit, wild pansy, rose Canina seed powder, and Radix polygoni multiflora, whereas all compositions are formulated with other components to serve as a cleanser, a moisturizer, a serum, a gel masque, an eye cream, a toner, or an exfoliant.

International Patent Application No. WO2017203529 provides compositions comprising a combination of cannabidiol (CBD) or a derivative thereof, and hyaluronic acid or a salt thereof; a phospholipid, and optionally a carrier, methods of using the compositions for treating inflammatory joint diseases, or pain or inflammation associated with such diseases, and methods for their preparation.

International Patent Application No. WO2018011808 provides self-emulsifying, high concentration and high dose cannabinoid compositions and formulations, to improve administration of cannabinoids and standardized marijuana extracts to patients.

Additional prior art documents include U.S. Patent Application Publication Nos. 20140302148, 20160374958, 20170044092, 20190111093, and 20190166903.

### SUMMARY OF THE INVENTION

The present disclosure provides a genus of drug-delivering molecular structures based on single-stranded hyaluronic acid (HA) carrying multiple copies of releasable cannabidiol (CBD) moieties.

Thus, according to an aspect of some embodiments of the present invention, there is provided a molecular structure comprising a hyaluronic acid (HA) moiety and a plurality of cannabidiol (CBD) moieties attached thereto via a biocleavable linking moiety.

In some embodiments, the structure includes at least two different types of biocleavable linking moieties.

In some embodiments, the biocleavable linking moiety is selected from the group consisting of amide, ester, carbonate, carbamate, thiocarbamate, sulfonamide, and phosphate.

In some embodiments, the HA moiety is a single-stranded HA moiety.

In some embodiments, the structure provided herein is characterized by an average CBD load of at least 5 wt. %.

According to another aspect of some embodiments of the present invention, there is provided a cosmetic composition that includes, as an active ingredient, the molecular structure provided herein, and a cosmetically acceptable carrier.

In some embodiments, the cosmetic composition provided herein is packaged in a packaging material and identified in print, in or on the packaging material, for use in the treatment of a skin condition.

According to another aspect of some embodiments of the present invention, there is provided a use of the molecular structure provided herein, in the preparation of a cosmetic composition.

In some embodiments, the cosmetic composition is identified for treating a skin condition.

According to another aspect of some embodiments of the present invention, there is provided a method of treating a skin condition in a subject in need thereof, the method is effected by administering to the subject an effective amount of the molecular structure provided herein or the cosmetic composition provided herein.

In some embodiments, the skin condition is selected from the group consisting of melasma, skin whitening, hyperpigmentation, Chadwick's sign, Linea alba, Perineal raphe, acne scarring, acne, liver spots, surgical scars, stretch marks and hair loss.

According to another aspect of some embodiments of the present invention, there is provided a process of preparing the molecular structure provided herein, which includes reacting CBD with a single-stranded HA to thereby obtain the molecular structure.

In some embodiments, the process further includes, prior to the reacting, modifying at least one functional group in the CBD to thereby obtain a reactive CBD, followed by reacting the reactive CBD with a single-stranded HA to thereby obtain the molecular structure.

In some embodiments, the process further includes, prior to the reacting, modifying at least one functional group in the HA to thereby obtain a reactive HA, followed by reacting the reactive HA with CBD to thereby obtain the molecular structure.

In some embodiments, the process further includes, prior to the reacting, modifying at least one functional group in the CBD to thereby obtain a reactive CBD, modifying at least one functional group in the HA to thereby obtain a reactive HA, followed by reacting the reactive HA with the reactive CBD to thereby obtain the molecular structure.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to cosmetics, and more particularly, but not exclusively, to a molecular structure based on multiple and releasable cannabidiol (CBD) moieties carried by hyaluronic acid (HA) as drug-delivery vehicle, and to uses thereof.

Before explaining at least one embodiment of the invention in detail, it is to be understood that the invention is not necessarily limited in its application to the details set forth in the following description or exemplified by the Examples. The disclosure is meant to encompass other embodiments or of being practiced or carried out in various ways.

While conceiving the present invention, the present inventors envisioned a molecular structure that is based on a single-stranded HA as a drug-delivery platform, which is chemically modified to carry multiple copies of releasable CBD moieties, linked to the HA strand via biocleavable linking moieties. HA lends itself for many chemical modifications via its reactive functional groups and can be used as a single-strand polysaccharide.

The present inventors have envisioned a molecular structure which, according to some embodiments, includes a single-stranded HA moiety, bearing at least one CBD moiety that is linked to HA and via biocleavable linkers, a.k.a. biodegradable moieties, thereby rendering HA a drug-delivery vehicle. The gist of the molecular structure is therefore a unique macromolecule that essentially has the physicomechanical and biochemical properties of HA, having one or more CBD moieties linked by biocleavable linkers, which can be used to target the bodily site, such as the skin, whereas upon biocleavage, releases CBD at the desired site.

### A Molecular Structure:

Thus, according to some embodiments of the present invention, there is provided a molecular structure, comprising a hyaluronic acid strand, which is referred to herein as an HA moiety, and a plurality of CBD moieties attached to single-stranded HA via a biocleavable linking moiety. In some embodiments, the molecular structure is devoid of crosslinked HA strands, and consists substantially on single-stranded HA moieties.

In general, the molecular structure provided herein comprises three structural elements, a hyaluronic acid strand, a plurality of CBD moieties attached thereto, and biocleavable linking moiety that link the first two elements. As used herein, the terms “moiety” and “residue”, used interchangeably, describe a portion of a molecule, and typically a major portion thereof, or a group of atoms pertaining to a specific function. The terms “moiety” and “residue” are used to refer to these elements in their bound form. In the context of the present invention, there terms are used to refer, for example, to a CBD molecule in its covalently bound form, as part of a molecular structure; in this example, CBD molecules or precursors thereof rare released from the molecular structure when the biocleavable linking moieties that link the CBD moiety to the molecular structure are cleaved.

Hyaluronic acid is presented in Scheme 1 below, and CBD is presented in Scheme 2 below, and both show potential functional groups that can be used for forming a biocleavable linking moiety.

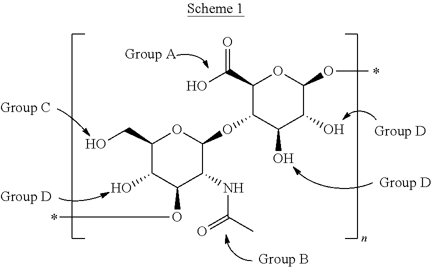

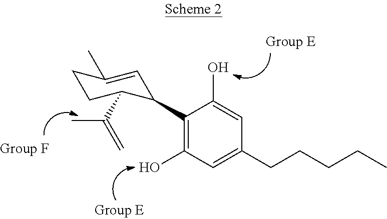

Schemes 1 and 2 show functional groups in HA and CBD, respectively, which are available for tethering and forming the biocleavable moiety. A list of exemplary conjugation options are listed below:
the carboxyl group in HA is referred to as Group A.
the N-acetamide group in HA is referred to as Group B.
the methanolyl group in HA is referred to as Group C.
any one of the hydroxyl groups in CBD is referred to as Group E.
the propylene-2-yl group in CBD is referred to as Group F.

In the molecular structures provided herein, any combination of Groups A-C in HA with Groups E and F in CBD form a biocleavable linking moiety.

Exemplary biocleavable moieties include, without limitation:
a biocleavable ester moiety that forms by attaching CBD to HA via Group E (hydroxyl) and Group A (carboxyl);
a biocleavable ester or ether moiety that forms by attaching CBD to HA via Group E (hydroxyl) and Group C (methanolyl);
a biocleavable carbonate moiety that forms by attaching CBD to HA via Group E (hydroxyl) and Group A (carboxyl);
a biocleavable carbonate moiety that forms by attaching CBD to HA via Group E (hydroxyl) and Group C (methanolyl);
a biocleavable carbamate moiety that forms by attaching CBD to HA via Group E (hydroxyl) and Group A (carboxyl);
a biocleavable carbamate moiety that forms by attaching CBD to HA via Group E (hydroxyl) and Group C (methanolyl);
a biocleavable thiocarbamate moiety that forms by attaching CBD to HA via Group E (hydroxyl) and Group A (carboxyl);
a biocleavable thiocarbamate moiety that forms by attaching CBD to HA via Group E (hydroxyl) and Group C (methanolyl);
a biocleavable sulfonamide moiety that forms by attaching CBD to HA via Group E (hydroxyl) and Group A (carboxyl);
a biocleavable sulfonamide moiety that forms by attaching CBD to HA via Group E (hydroxyl) and Group C (methanolyl);
a biocleavable phosphate moiety that forms by attaching CBD to HA via Group E (hydroxyl) and Group A (carboxyl);
a biocleavable phosphate moiety that forms by attaching CBD to HA via Group E (hydroxyl) and Group A (carboxyl);
In some embodiments, the molecular structure is also capable of releasing a precursor of CBD, which is oftentimes referred to as a prodrug of CBD. The term “prodrug” refers to an agent, which is converted into a bioactive agent (the active parent drug) in vivo. In essence, the entire molecular structures presented herein constitute a form of a prodrug, as CBD moieties, which are designed for release as bioactive agents in a controllable manner, are linked thereto. Prodrugs are typically useful for facilitating and/or targeting the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of a bioactive agent in vivo. An example, without limitation, of a prodrug would be a bioactive agent, according to some embodiments of the present invention, having one or more carboxylic acid moieties, which is administered as an ester (the “prodrug”). Such a prodrug is hydrolyzed in vivo, to thereby provide the free bioactive agent (CBD). The selected ester may affect both the solubility characteristics and the hydrolysis rate of the prodrug. A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)acyl (e.g., acetyl) group to form an ester group, and/or any one or more of the carboxylic acid groups of the compound is modified by an alkoxy or aryloxy group, optionally (C₁₋₄)alkoxy (e.g., methyl, ethyl) group to form an ester group. Exemplary prodrugs of cannabidiol (CBD) can be found, without limitation, in documents such as U.S. Pat. No. 8,293,786.

In some embodiments, the HA stand and/or the CBD molecule of the molecular structure provided herein, undergo modification of one of more their native functional groups in order to increase the efficiency of attachment, or introduce a functionality to the HA strand that can react with compatible function group in CBD. When forming a part of the molecular structure, these modifications form a part of the linking moiety that is afforded during the CBD loading reaction. Exemplary functional group modifications that result in the introduction of an amide functionality to the HA moiety include, without limitation, Gly, β-Ala, GABA, 3-Amino-2, 2-dimethyl-propionic acid, sarcosine, and NH₂-PEG4-Propionic acid. Exemplary functional group modifications that result in the modification of a carboxyl in the HA moiety into an ester functionality include, without limitation, hydroxyacetic acid, hydroxypropanoic acid, and hydroxybenzoic acid. Exemplary functional group modifications that result in the modification of a hydroxyl in the HA moiety into an ester functionality include, without limitation, succinic acid, glutaric acid, adipic acid, and phthalic acid. Exemplary functional group modifications that result in the modification of a carboxyl in the HA moiety into a hydrazide functionality include, without limitation, glycine hydrazide, alanine hydrazide, and β-alanine hydrazide.

Since the formation of a plurality of CBD moieties on a large polymeric entity like HA is not a deterministic process but a statistical process, the molecular structures can be characterized also by the percentage of CBD moieties loaded on the HA strand. An assessment of the percentage of elements present in the structure can be effected, for example, by submitting a batch of the molecular structure, according to some embodiments of the present invention, to total degradation by hyaluronidase in D20 and determination of the percentage of the CBD-load by proton NMR comparing the area under the relevant peaks. A similar approach can be effected for determination of the percentage of CBD-load on the molecular structure, by submitting the same to total cleavage of all linking moieties, and following detectable markers for CBD released from the molecular structure. For example, 10% loading of CBD means area under the peak associated with HA is 10 times greater than the area under the peak associated with CBD.

In some embodiments, the average CBD-load in the molecular structure is greater than 5 wt. %, 10 wt. %, 20 wt. %, 30 wt. %, 40 wt. %, 50 wt. %, or greater than 60 wt. %. In some embodiments, the average CBD-load ranges 5-60 wt. %, 10-40 wt. %, 20-50 wt. %, or 30-60 wt. %.

For any of the embodiments described herein, the molecular structures described herein may be in a form of a salt, for example, a cosmetically and/or pharmaceutically acceptable salt. As used herein, the phrase “cosmetically and/or pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound.

In the context of some of the present embodiments, a cosmetically and/or pharmaceutically acceptable salt of the compounds described herein may optionally be a base addition salt comprising at least one acidic (e.g., carboxylic acid) group of the compound which is in a negatively charged form, e.g., wherein the acidic group is deprotonated, in combination with at least one counter-ion, derived from the selected base, that forms a pharmaceutically acceptable salt.

The base addition salts of the compounds described herein may therefore be complexes formed between one or more acidic groups of the drug and one or more equivalents of a base.

The base addition salts may include a variety of organic and inorganic counter-ions and bases, such as, but not limited to, sodium (e.g., by addition of NaOH), potassium (e.g., by addition of KOH), calcium (e.g., by addition of Ca(OH)₂, magnesium (e.g., by addition of Mg(OH)₂), aluminum (e.g., by addition of Al(OH)₃ and ammonium (e.g., by addition of ammonia). Each of these acid addition salts can be either a mono-addition salt or a poly-addition salt, as these terms are defined herein.

Depending on the stoichiometric proportions between the charged group(s) in the compound and the counter-ion in the salt, the acid or base additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

Further, each of the compounds described herein, including the salts thereof, can be in a form of a solvate or a hydrate thereof.

The compounds described herein can be used as polymorphs and the present embodiments further encompass any isomorph of the compounds and any combination thereof.

The present embodiments further encompass any enantiomers, prodrugs, solvates, hydrates and/or pharmaceutically acceptable salts of the molecular structures described herein and methods, compositions and uses utilizing enantiomers, diastereomers, prodrugs, solvates, hydrates and/or pharmaceutically acceptable salts of the molecular structures described herein.

The term “solvate” refers to a complex of variable stoichiometric (e.g., di-, tri-, tetra-, penta-, hexa-, and so on), which is formed by a solute (the molecular structures described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

### Biocleavable Inking Moiety:

As used herein, the term “linking moiety” describes a chemical moiety (a group of covalently-bonded atoms or a single, or a double, or a triple covalent bond) that links CBD moieties to HA via one or more covalent bonds. A linking moiety may include atoms that form a part of one or both of the chemical moieties it links, and/or include atoms that do not form a part of one or both of the chemical moieties it links. For example, a peptide bond (amide) linking moiety that links two chemical moieties includes at least a nitrogen atom and a hydrogen atom from one bioactive agent moiety and at least a carboxyl of the other bioactive agent moiety. In general, the linking moiety can be formed during a chemical reaction, such that by reacting two or more reactive groups, the linking moiety is formed as a new chemical entity which can comprise a bond (between two atoms), or one or more bonded atoms. Alternatively, the linking moiety can be an independent chemical moiety comprising two or more reactive groups to which the reactive groups of other compounds can be attached, either directly or indirectly, as is detailed hereinunder.

The positions at which the bioactive agent is linked to the molecular structure presented herein are generally selected such that once cleaved off the molecular structure, any remaining moiety stemming from the linking moiety on HA and/or CBD, if at all, does not substantially preclude its biological activity (mechanism of biological activity). According to some embodiments of the present invention, the linking moieties are form such that the biological activity of CBD, once released from the molecular structure, is not abolished and remains substantially the same as the biological activity of pristine CBD. According to some embodiments of the present invention, the linking moiety is such that once CBD is released from the molecular structure, it is a pristine CBD molecule or a prodrug (precursor) thereof.

In some embodiments, the term “linking moiety” is defined so as not to encompass a moiety that once the linking moiety is cleaved, standalone molecule is released. This limitation excludes linking moiety that releases upon cleavage standalone molecules such water molecules, gas molecules, small organic ions, such as acetate, small inorganic ions such as hydroxide, and the likes. In such embodiments, the molecular structure may be regarded as one that does not release non-bioactive agents.

As used herein, the words “link”, “linked”, “linkage” “linker”, “bound” or “attached”, are used interchangeably herein and refer to the presence of at least one covalent bond between species, unless specifically noted otherwise.

As stated hereinabove, a linking moiety can be formed during a chemical reaction, such that by reacting two or more reactive groups. The phrase “reactive group”, as used herein, refers to a chemical group that is capable of undergoing a chemical reaction that typically leads to the formation a covalent bond. Reactive groups include Groups A-F presented hereinabove. Chemical reactions that lead to a bond formation include, for example, cycloaddition reactions (such as the Diels-Alder's reaction, the 1,3-dipolar cycloaddition Huisgen reaction, and the similar “click reaction”), condensations, nucleophilic and electrophilic addition reactions, nucleophilic and electrophilic substitutions, addition and elimination reactions, alkylation reactions, rearrangement reactions and any other known organic reactions that involve a reactive group.

Representative examples of reactive groups include, without limitation, acyl halide, aldehyde, alkoxy, alkyne, amide, amine, aryloxy, azide, aziridine, azo, carbamate, carbonyl, carboxyl, carboxylate, cyano, diene, dienophile, epoxy, guanidine, guanyl, halide, hydrazide, hydrazine, hydroxy, hydroxylamine, imino, isocyanate, nitro, phosphate, phosphonate, sulfinyl, sulfonamide, sulfonate, thioalkoxy, thioaryloxy, thiocarbamate, thiocarbonyl, thiohydroxy, thiourea and urea, as these terms are defined hereinafter.

Biocleavable linking moieties, according to some embodiments of the present invention, include without limitation, amide, ester, ether, carbonate, carbamate, thiocarbamate, sulfonamide, and phosphate.

Additional non-limiting examples of linking moieties, according to some embodiments of the present invention, include, amide, carbamate, carbonate, lactone, lactam, carboxylate, ester, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thioacetal, thioketal, phosphate ester, and the like. Other linking moieties are defined hereinbelow, and further other linking moieties are contemplated within the scope of the term as used herein.

According to some embodiments, the linking moiety is selected from the group consisting of:

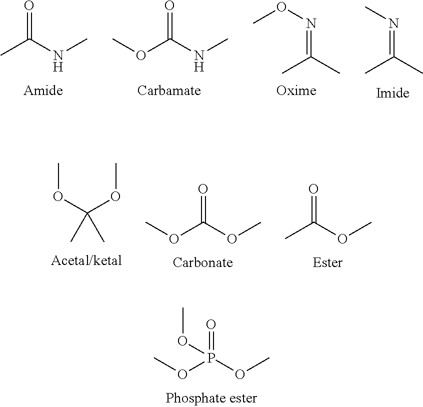

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

As used herein, the terms “amine” or “amino”, describe both a —NR′R″ end group and a —NR′— linking moiety, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydrogen, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine, as these terms are defined herein.

The term “alkyl” describes a saturated aliphatic hydrocarbon including straight chain (unbranched) and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking moiety, as this phrase is defined hereinabove, which connects two or more moieties via at least two carbons in its chain. When an alkyl is a linking moiety, it is also referred to herein as “alkylene”, e.g., methylene, ethylene, propylene, etc.

The term “alkenyl” describes an unsaturated alkyl, as defined herein, having at least two carbon atoms and at least one carbon-carbon double bond. The alkenyl may be substituted or unsubstituted by one or more substituents, as described for alkyl hereinabove.

The terms “alkynyl” or “alkyne”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings that share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking moiety, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking moiety, as this phrase is defined hereinabove, connecting two or more moieties at two or more positions thereof. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking moiety, as this term is defined hereinabove, connecting two or more moieties at two or more positions thereof. Preferably, the aryl is phenyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking moiety, as this phrase is defined hereinabove, connecting two or more moieties at two or more positions thereof. Representative examples are pyridine, pyrrole, oxazole, indole, purine and the like.

The term “alkaryl” describes an alkyl, as defined herein, which is substituted by one or more aryl or heteroaryl groups. An example of alkaryl is benzyl.

The term “amine-oxide” describes a —N(OR′)(R″) or a —N(OR′)— group, where R′ and R″ are as defined herein. This term refers to a —N(OR′)(R″) group in cases where the amine-oxide is an end group, as this phrase is defined hereinabove, and to a —N(OR′)— group in cases where the amine-oxime is an end group, as this phrase is defined hereinabove.

As used herein, the term “acyl” refers to a group having the general formula —C(═O)R′, —C(═O)OR′, —C(═O)—O—C(═O)R′, —C(═O)SR′, —C(═O)N(R′)₂, —C(═S) R′, —C(═S)N(R′)₂, and —C(═S) S(R′), —C(═NR′)R″, —C(═NR′)OR″, —C(═NR′)SR″, and —C(═NR′)N(R″)₂, wherein R′ and R″ are each independently hydrogen, halo, substituted or unsubstituted hydroxyl, substituted or unsubstituted thiol, substituted or unsubstituted amine, substituted or unsubstituted acyl, cyclic or acyclic, substituted or unsubstituted, branched or unbranched aliphatic, cyclic or acyclic, substituted or unsubstituted, branched or unbranched heteroaliphatic, cyclic or acyclic, substituted or unsubstituted, branched or unbranched alkyl, cyclic or acyclic, substituted or unsubstituted, branched or unbranched alkenyl, substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thioxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. Unless otherwise specified, heteroaliphatic groups contain 1-6 carbon atoms wherein 1-3 carbon atoms are optionally and independently replaced with heteroatoms selected from oxygen, nitrogen and sulfur. In some embodiments, heteroaliphatic groups contain 1-4 carbon atoms, wherein 1-2 carbon atoms are optionally and independently replaced with heteroatoms selected from oxygen, nitrogen and sulfur. In yet other embodiments, heteroaliphatic groups contain 1-3 carbon atoms, wherein 1 carbon atom is optionally and independently replaced with a heteroatom selected from oxygen, nitrogen and sulfur. Suitable heteroaliphatic groups include, but are not limited to, linear or branched, heteroalkyl, heteroalkenyl, and heteroalkynyl groups.

The term “halo” describes fluorine, chlorine, bromine or iodine substituent.

The term “halide” describes an anion of a halogen atom, namely F⁻, Cl⁻Br and I⁻.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking moiety, as these phrases are defined hereinabove, where R′ is as defined hereinabove.

The term “thiosulfate” describes a —O—S(═S)(═O)—OR′ end group or a —O—S(═S)(═O)—O— linking moiety, as these phrases are defined hereinabove, where R′ is as defined hereinabove.

The term “sulfite” describes an —O—S(═O)—O—R′ end group or a —O—S(═O)—O— group linking moiety, as these phrases are defined hereinabove, where R′ is as defined hereinabove.

The term “thiosulfite” describes a —O—S(═S)—O—R′ end group or an —O—S(═S)—O— group linking moiety, as these phrases are defined hereinabove, where R′ is as defined hereinabove.

The term “sulfinate” or “sulfinyl” describes a —S(═O)—OR′ end group or an —S(═O)—O— group linking moiety, as these phrases are defined hereinabove, where R′ is as defined hereinabove.

The terms “solfoxide” or “sulfinyl” describe a —S(═O)R′ end group or an —S(═O)— linking moiety, as these phrases are defined hereinabove, where R′ is as defined hereinabove.

The term “sulfonate” or “sulfonyl” describes a —S(═O)₂—R′ end group or an —S(═O)₂— linking moiety, as these phrases are defined hereinabove, where R′ is as defined herein.

The term “S-sulfonamide” describes a —S(═O)₂—NR′R″ end group or a —S(═O)₂—NR′— linking moiety, as these phrases are defined hereinabove, with R′ and R″ as defined herein.

The term “N-sulfonamide” describes an R'S(═O)₂—NR″— end group or a —S(═O)₂—NR′— linking moiety, as these phrases are defined hereinabove, where R′ and R″ are as defined herein.

The term “disulfide” refers to a —S—SR′ end group or a —S—S— linking moiety, as these phrases are defined hereinabove, where R′ is as defined herein.

The term “phosphate” describes an —O—P(═O)₂(OR′) end or reactive group or a —O—P(═O)₂(O)— linking moiety, as these phrases are defined hereinabove, with R′ as defined herein.

The term “phosphonate” describes a —P(═O)(OR′)(OR″) end or reactive group or a —P(═O)(OR′)(O)— linking moiety, as these phrases are defined hereinabove, with R′ and R″ as defined herein.

The term “thiophosphonate” describes a —P(═S)(OR′)(OR″) end group or a —P(═S)(OR′)(O)— linking moiety, as these phrases are defined hereinabove, with R′ and R″ as defined herein.

The term “carbonyl” or “carbonate” as used herein, describes a —C(═O)—R′ end group or a —C(═O)— linking moiety, as these phrases are defined hereinabove, with R′ as defined herein.

The term “thiocarbonyl” as used herein, describes a —C(═S)—R′ end group or a —C(═S)— linking moiety, as these phrases are defined hereinabove, with R′ as defined herein.

The term “oxo” as used herein, described a ═O end group.

The term “thioxo” as used herein, described a ═S end group.

The term “oxime” describes a ═N—OH end group or a ═N—O— linking moiety, as these phrases are defined hereinabove.

The term “hydroxyl” describes a —OH group.

As used herein, the term “aldehyde” refers to an —C(═O)—H group.

The term “acyl halide” describes a —(C═O)R″ group wherein R″″ is halo, as defined hereinabove.

The term “alkoxy” as used herein describes an —O-alkyl, an —O-cycloalkyl, as defined hereinabove. The ether group —O— is also a possible linking moiety.

The term “aryloxy” describes both an —O-aryl and an —O-heteroaryl group, as defined herein.

The term “disulfide” as used herein describes an —S—S— linking moiety, which in some cases forms between two thiohydroxyl groups.

The terms “thio”, “sulfhydryl” or “thiohydroxyl” as used herein describe an —SH group.

The term “thioalkoxy” or “thioether” describes both a —S-alkyl group, and a —S— cycloalkyl group, as defined herein. The thioether group —S— is also a possible linking moiety.

The term “thioaryloxy” describes both a —S-aryl and a —S-heteroaryl group, as defined herein. The thioarylether group —S-aryl- is also a possible linking moiety.

The term “cyano” or “nitrile” describes a —C═N group.

The term “isocyanate” describes an —N═C═O group.

The term “nitro” describes an —NO₂ group.

The term “carboxylate” or “ester”, as used herein encompasses C-carboxylate and O-carboxylate.

The term “C-carboxylate” describes a —C(═O)—OR′ end group or a —C(═O)—O— linking moiety, as these phrases are defined hereinabove, where R′ is as defined herein.

The term “O-carboxylate” describes a —OC(—O)R′ end group or a —OC(═O)— linking moiety, as these phrases are defined hereinabove, where R′ is as defined herein.

The term “thiocarboxylate” as used herein encompasses “C-thiocarboxylate and O-thiocarboxylate.

The term “C-thiocarboxylate” describes a —C(═S)—OR′ end group or a —C(═S)—O— linking moiety, as these phrases are defined hereinabove, where R′ is as defined herein.

The term “O-thiocarboxylate” describes a —OC(═S)R′ end group or a —OC(═S)— linking moiety, as these phrases are defined hereinabove, where R′ is as defined herein.

The term “carbamate” as used herein encompasses N-carbamate and O-carbamate.

The term “N-carbamate” describes an R″OC(═O)—NR′— end group or a —OC(═O)—NR′— linking moiety, as these phrases are defined hereinabove, with R′ and R″ as defined herein.

The term “O-carbamate” describes an —OC(═O)—NR′R″ end group or an —OC(═O)—NR′— linking moiety, as these phrases are defined hereinabove, with R′ and R″ as defined herein.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking moiety, as these phrases are defined hereinabove, with R′ and R″ as defined herein.

The term “N-thiocarbamate” describes an R″OC(═S)NR′— end group or a —OC(═S)NR′— linking moiety, as these phrases are defined hereinabove, with R′ and R″ as defined herein.

The term “dithiocarbamate” as used herein encompasses N-dithiocarbamate and S-dithiocarbamate.

The term “S-dithiocarbamate” describes a —SC(═S)—NR′R″ end group or a —SC(═S)NR′— linking moiety, as these phrases are defined hereinabove, with R′ and R″ as defined herein.

The term “N-dithiocarbamate” describes an R″SC(═S)NR′— end group or a —SC(═S)NR′— linking moiety, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking moiety, as these phrases are defined hereinabove, where R′ and R″ are as defined herein and R″ is as defined herein for R′ and R″.

The term “thiourea”, which is also referred to herein as “thioureido”, describes a —NR′—C(═S)—NR″R′″ end group or a —NR′—C(═S)—NR″— linking moiety, with R′, R″ and R′″ as defined herein.

The term “amide” as used herein encompasses C-amide and N-amide.

The term “C-amide” describes a —C(═O)—NR′R″ end group or a —C(═O)—NR′— linking moiety, as these phrases are defined hereinabove, where R′ and R″ are as defined herein.

The term “N-amide” describes a R′C(═O)—NR″— end group or a R′C(—O)—N— linking moiety, as these phrases are defined hereinabove, where R′ and R″ are as defined herein.

The term “imine”, which is also referred to in the art interchangeably as “Schiff-base”, describes a —N═CR′— linking moiety, with R′ as defined herein or hydrogen. As is well known in the art, Schiff bases are typically formed by reacting an aldehyde or a ketone and an amine-containing moiety such as amine, hydrazine, hydrazide and the like, as these terms are defined herein. The term “aldimine” refers to a —CH═N— imine which is derived from an aldehyde. The term “ketimine” refers to a —CR′═N— imine which is derived from a ketone.

The term “hydrazone” refers to a —R′C═N—NR″— linking moiety, wherein R′ and R″ are as defined herein.

The term “semicarbazone” refers to a linking moiety which forms in a condensation reaction between an aldehyde or ketone and semicarbazide. A semicarbazone linking moiety stemming from a ketone is a —R′C—NNR″C(═O)NR′″—, and a linking moiety stemming from an aldehyde is a —CR′═NNR″C(═O)NR′″—, wherein R′ and R″ are as defined herein and R″ or as defined for R′.

As used herein, the term “lactone” refers to a cyclic ester, namely the intra-condensation product of an alcohol group —OH and a carboxylic acid group —COOH in the same molecule.

As used herein, the term “lactam” refers to a cyclic amide, as this term is defined herein. A lactam with two carbon atoms beside the carbonyl and four ring atoms in total is referred to as a β-lactam, a lactam with three carbon atoms beside the carbonyl and five ring atoms in total is referred to as a γ-lactam, a lactam with four carbon atoms beside the carbonyl and six ring atoms in total is referred to as a δ-lactam, and so on.

The term “guanyl” describes a R′R″NC(═N)— end group or a —R′NC(═N)— linking moiety, as these phrases are defined hereinabove, where R′ and R″ are as defined herein.

The term “guanidine” describes a —R′NC(═N)—NR″R′″ end group or a —R′NC(═N)—NR″— linking moiety, as these phrases are defined hereinabove, where R′, R″ and R′″ are as defined herein.

The term “hydrazine” describes a —NR′—NR″R′″ end group or a —NR′—NR″— linking moiety, as these phrases are defined hereinabove, with R′, R″, and R″ as defined herein.

As used herein, the term “hydrazide” describes a —C(═O)—NR′—NR″R′″ end group or a —C(═O)—NR′—NR″— linking moiety, as these phrases are defined hereinabove, where R′, R″ and R′″ are as defined herein.

The term “hydroxylamine”, as used herein, refers to either a —NHOH group or a —ONH₂.

As used herein, the terms “azo” or “diazo” describe a —N═N—R′ end group or a —N═N— linking moiety, as these phrases are defined hereinabove, where R′ is as defined herein.

As used herein, the term “azido” described a —N═N⁺═N—(—N₃) end group.

The term “triazine” refers to a heterocyclic ring, analogous to the six-membered benzene ring but with three carbons replaced by nitrogen atoms. The three isomers of triazine are distinguished from each other by the positions of their nitrogen atoms, and are referred to as 1,2,3-triazine, 1,2,4-triazine, and 1,3,5-triazine. Other aromatic nitrogen heterocycles include pyridines with 1 ring nitrogen atom, diazines with 2 nitrogen atoms in the ring and tetrazines with 4 ring nitrogen atoms.

The term “triazole” refers to either one of a pair of isomeric chemical compounds with molecular formula C₂H₃N₃, having a five-membered ring of two carbon atoms and three nitrogen atoms, namely 1,2,3-triazoles and 1,2,4-triazoles.

The term “aziridine”, as used herein, refers to a reactive group which is a three membered heterocycle with one amine group and two methylene groups, having a molecular formula of —C₂H₃NH.

As used herein, the term “thiohydrazide” describes a —C(═S)—NR′—NR″R′″ end group or a —C(═S)—NR′—NR″— linking moiety, as these phrases are defined hereinabove, where R′, R″ and R′″ are as defined herein.

As used herein, the term “methyleneamine” describes an —NR′—CH₂—CH═CR″R″ end group or a —NR′—CH₂—CH═CR″— linking moiety, as these phrases are defined hereinabove, where R′, R″ and R′″ are as defined herein.

The term “diene”, as used herein, refers to a —CR′═CR″—CR′″═CR′″— group, wherein R′ as defined hereinabove, and R″, R″ and R′″ are as defined for R′.

The term “dienophile”, as used herein, refers to a reactive group that reacts with a diene, typically in a Diels-Alder reaction mechanism, hence a dienophile is typically a double bond or an alkenyl.

The term “epoxy”, as used herein, refers to a reactive group which is a three membered heterocycle with one oxygen and two methylene groups, having a molecular formula of —C₂H₃O.

The phrase “covalent bond”, as used herein, refers to one or more pairs of electrons that are shared between atoms in a form of chemical bonding.

According to some embodiments of the present invention, some linking moieties result from a reaction between two reactive groups. Alternatively, a desired linking moiety is first generated and a bioactive agent and/or a spacer moiety are attached thereto.

### Linking Moiety Lability:

In some embodiments, each of the linking moieties in the molecular structure is the same biocleavable linking moiety, namely, all the CBD moieties are attached to HA by the same linking moiety.

In some embodiments, the molecular structure comprises CBD moieties attached to HA via more than one type of linking moieties. In some of these embodiments, each type of the linking moieties is also characterized by a different biocleavage condition.

The term “biocleavage” refers to a biochemical reaction that causes the linking moiety to break/dissociate. In the context of embodiments of the present invention, biocleavage is typically mediated by biomolecules (e.g., enzymes, RNA and the likes) in an organism or an organ thereof, whereas each such mediator is active or more active under certain conditions, including location in the organism (cell, tissue, organ), temperature, pH, ionic strength, light and other reaction effectors, as these are known in the art. Linking moieties having different biocleavage condition allow a differential release of CBD at different locations in the subject, and/or different times, and/or under otherwise different physiological conditions.

A linking moiety that is stable at physiological conditions, namely the linking moiety does not disintegrate for the duration of exposure to the physiological environment in the bodily site, is referred to herein a “biostable linking moiety”. An exemplary biostable linking moiety is a triazole-based linking moiety. It is noted that biostability is also a relative term, meaning that a biostable linking moiety takes longer to break or requires certain cleavage conditions which hare less frequently encountered by the molecular structure when present in physiological conditions.

In some embodiments of the present invention, the linking moieties in the molecular structure provided herein are all biocleavable. In the context of some embodiments of the present invention, biocleavable linking moieties are selected so as to break and release a plurality of CBD molecules or precursors thereof at certain conditions, referred to herein as “drug-releasing conditions” or “biocleavage conditions”.

According to some embodiments of the present invention, some of the linking moieties are biocleavable-linking moieties. As used herein, the terms “biocleavable” and “biodegradable” are used interchangeably to refer to moieties that degrade (i.e., break and/or lose at least some of their covalent structure) under physiological or endosomal conditions. Biodegradable moieties are not necessarily hydrolytically degradable and may require enzymatic action to degrade.

As used herein, the terms “biocleavable moiety” or “biodegradable moiety” describe a chemical moiety, which undergoes cleavage in a biological system such as, for example, the digestive system of an organism or a metabolic system in a living cell.

In some embodiments, biocleavable linking moieties are selected according to their susceptibility to certain enzymes that are likely to be present at the targeted bodily site or at any other bodily site where cleavage is intended, thereby defining the cleavage conditions.

Representative examples of biocleavable moieties include, without limitation, amides, carboxylates, carbamates, phosphates, hydrazides, thiohydrazides, disulfides, epoxides, peroxo and methyleneamines. Such moieties are typically subjected to enzymatic cleavages in a biological system, by enzymes such as, for example, hydrolases, amidases, kinases, peptidases, phospholipases, lipases, proteases, esterases, epoxide hydrolases, nitrilases, glycosidases and the like.

For example, hydrolases (EC number beginning with 3) catalyze hydrolysis of a chemical bond according to the general reaction scheme A-B+H₂O→A-OH+B-H. Ester bonds are cleaved by sub-group of hydrolases known as esterases (EC number beginning with 3.1), which include nucleases, phosphodiesterases, lipases and phosphatases. Hydrolases having an EC number beginning with 3.4 are peptidases, which act on peptide bonds.

Additional information pertaining to enzymes, enzymatic reactions, and enzyme-linking moiety correlations can be found in various publically accessible sources, such as Bairoch A., “The ENZYME database in 2000”, Nucleic Acids Res, 2000, 28, pp. 304-305.

In some embodiments, certain linking moieties are selected to be more labile than other linking moieties present in the molecular structure. By “more labile”, it is meant that some of the linking moieties have a higher tendency to break at given cleavage conditions compared to other linking moieties. In some embodiments, the linking moieties are selected according to a certain lability hierarchy that allows the design of a particular drug-releasing profile, wherein the order and the rate of drug release is controllable according to the lability hierarchy. In the context of some embodiment of the invention, the more labile linking moieties, higher in the lability hierarchy will break first and at a higher rate than those lower in the lability hierarchy. The ability to select linking moieties according to their lability hierarchy provides molecular structures with differential CBD-releasing profiles, according to some embodiments of the present invention.

The selection of the linking moieties according to lability hierarchy is determined according to the cleavage conditions, which the molecular structure is expected to experience once it is administered into a living cell/tissue/organ (collectively referred to herein as a “bodily site”). Cleavage conditions include the chemical and physical conditions that are present in the bodily site, such as temperature, pH, the presence of reactive species and the presence of enzymes, all of which can cause a given linking moiety to break and release CBD or precursors thereof.

For example, some linking moieties are more labile (susceptible to) in higher temperatures, while others are susceptible to higher or lower pH values compared to other linking moieties. In such cases, a molecular structure which is design to target a bodily site that is characterized by a localized pH value compared to its surroundings, an acid-labile or an H⁺-labile linking moiety is advantageously selected to release CBD.

In some embodiments, each of the linking moieties is characterized by a given cleavage condition, and any one of the linking moieties is selected such that at least one thereof is different than one-another, based on the cleavage condition thereof.

### Applications:

Since the molecular structures presented herein carry, deliver and controllably release a load of CBD molecules or precursors thereof, the molecular structures can be used to treat various medical conditions, and in particular, dermatologic conditions. The molecular structures presented herein can therefore be used as an active ingredient in a variety of pharmaceutical and cosmetic compositions, and in the preparation of a variety of medicaments.

Accordingly there is provided a pharmaceutical composition and/or a cosmetic composition that includes, as an active ingredient, the molecular structure provided herein, according to embodiments of the present invention, and a pharmaceutically and/or cosmetically acceptable carrier.

Similarly, there is provided a use of the molecular structure, according to embodiments of the present invention, in the preparation of a pharmaceutical and/or a cosmetic medicament.

Also provided herein is a method of treating a skin condition in a subject in need thereof, which includes administering to the subject an effective amount of the molecular structure, according to embodiments of the present invention.

According to some embodiments of the present invention, the pharmaceutical composition or medicament, are used to treat a medical or a dermatologic or a cosmetic condition, and more preferably a dermatologic/skin condition. In some embodiments, the medical condition is treatable by CBD. In some embodiments, the presence of HA and CBD exerts a synergistic effect, namely the beneficial effect of the combined elements of the molecular structure exert a greater beneficial effect than the combined effect of each of the elements administered alone.

As used herein, the phrase “effective amount” describes an amount of a molecular structure being administered, which will relieve to some extent one or more of the symptoms of the dermatologic/skin condition being treated. In the context of the present embodiments, the phrase “effective amount” describes an amount of a molecular structure being administered and/or re-administered, which will relieve to some extent one or more of the symptoms of the dermatologic/skin condition being treated by being at a level that is beneficial to the target cell(s) or tissue(s), and effects a notable betterment of the skin condition.

In the context of embodiments of the present invention, the effective amount may refer to the molecular structure as a whole or to the amount of CBD releasably attached thereto. The efficacy of CBD, including the molecular structures presented herein, can be determined by several methodologies known in the art.

According to another aspect of embodiments of the present invention, any one of the molecular structures described herein is identified for use in treating a subject diagnosed with a skin condition treatable by CBD linked and controllably releasable from the molecular structure.

According to another aspect of embodiments of the present invention, there is provided a use of any of the molecular structures described herein as a medicament. In some embodiments, the medicament is for treating a subject diagnosed with a skin condition treatable by CBD linked and controllably releasable from the molecular structure.

In any of the methods and uses described herein, the molecular structure can be administered as a part of a pharmaceutical or cosmetic composition, which further comprises a pharmaceutically and/or cosmetically acceptable carrier, as known in the art. The carrier is selected suitable to the selected route of administration.

The molecular structures presented herein can be administered via several administration route, including, but not limited to, topically, subcutaneous, and orally. In some preferred embodiments, the molecular structure provided herein is administered using percutaneous and minimally invasive tools and methods, typically used to treat numerous dermatologic/skin conditions.

As a composition for treating various skin conditions, the molecular structure is particularly useful for topical and/or subcutaneous administration. In some embodiments, the preferred mode of administration is microneedling, also known as collagen induction therapy, which is a process involving repetitive and shallow puncturing of the skin with sterilized microneedles. Microneedling is typically effected by use of a dermaroller, whereas the cosmetic composition that includes the presently disclosed molecular structure, is applied on the skin area to be treated, and the dermaroller is used over this skin area. Alternatively, a dermaroller can be laced or loaded with a composition comprising the molecular structure provided herein. Further alternatively, microneedling for introduction of the molecular structure presented herein is effected by a syringe equipped with a small subcutaneous needle for shallow (2-3 mm) skin penetration.

According to some embodiments of the present invention, the molecular structure can be co-administered with one or more known drugs, compositions, medicaments and drugs suitable for treating a dermatologic/skin condition.

According to some embodiments, the composition comprising the molecular structure provided herein is packaged in a packaging material and identified in print, in or on the packaging material, for use in the treatment of a dermatologic/skin condition treatable by CBD linked and controllably releasable from the molecular structure, including CBD and HA.

As used herein the term “composition” or the term “medicament” refer to a preparation of the molecular structures presented herein, with other chemical components such as pharmaceutically/dermatologically/cosmetically acceptable and suitable carriers and excipients, and optionally with additional bioactive agents or compositions comprising the same. The purpose of a pharmaceutical or cosmetic composition is to facilitate administration of the molecular structure to a subject.

Hereinafter, the phrase “pharmaceutically and/or cosmetically acceptable carrier” refers to a carrier or a diluent that does not cause significant irritation to the subject of the treatment and does not abrogate the biological activity and properties of the administered molecular structure. Examples, without limitations, of pharmaceutically and/or cosmetically acceptable carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a molecular structure. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, PA, latest edition, which is incorporated herein by reference.

The dosage may vary depending upon the dosage form employed and the route of administration utilized. The exact formulation, route of administration and dosage can be chosen by the individual caretaker in view of the subject's condition. (See e.g., Fingl et al., 1975, in “The Pharmacological Basis of Therapeutics”, Ch. 1 p. 1). In general, the dosage is related to the efficacy of the active ingredient and the severity of the skin condition. The amount of a composition to be administered will, of course, be dependent on the subject being treated, the severity of the affliction, the manner of administration, the judgment of the prescribing caretaker, etc.

### Medical Conditions:

The molecular structure presented herein can be used to treat dermatologic/skin conditions that are treatable by administration of a bioactive agent (drug) that is releasable form therefrom. Dermatologic/skin conditions can be caused by environmental factors, age and genetic factors, cancer, autoimmunity, and microorganisms.

Skin diseases and conditions, including nail and hair, are caused by viruses, rickettsiae, bacteria, fungi, and parasites. In some embodiments of the present invention, the skin condition treatable by the molecular structure provided herein is associated with an infection caused by a microorganism, including a viral infection, a bacterial infection, a yeast infection, a fungal infection, a protozoan infection, a parasite-related infection and the like.

Skin/dermatological conditions associated with a microorganism include, without limitation, impetigo, cellulitis and erysipelas, staphylococcal scalded skin syndrome, folliculitis, erysipeloid, pitted keratolysis, erythrasma, trichomycosis, intertrigo, acute infectious eczematoid dermatitis, pseudofolliculitis of the beard, toe web infection, skin tuberculosis (localized form), Mycobacterium marinum skin disease, Mycobacterium ulcerans skin disease, actinomycetoma, and actinomycosis.

In some embodiments, the molecular structure is design to release bioactive agents that are beneficial in the treatment of skin conditions, such as, but not limited to, melasma, skin whitening, hyperpigmentation, Chadwick's sign, Linea alba, Perineal raphe, acne scarring, acne, liver spots, surgical scars, stretch marks, and hair loss.

### Preparation of Molecular Structures:

As can be appreciated form the diversity and complexity of the scope of embodiments of the present invention, the molecular structure provided herein can be afforded via various synthetic approaches. For example, the construction of the molecular structure can begin by reacting CBD and HA in the presence of suitable reagents that promote and/or participate in the formation of a linking moiety between one of the reactive groups on both HA and CBD, thereby attaching a plurality of CBD moieties to single-stranded HA.

Alternatively, HA and/or CBD are modifies at one or more of their native reactive groups to afford one or more modified reactive groups, followed by reacting the modified HA and/or CBD with one-another.

In some embodiments, HA is partially loaded with CBD via a given type of linking moiety, and this partially loaded molecular structure is further reacted with CBD under different conditions and reaction to further load more CBD moieties on the partially loaded molecular structure, thereby forming a molecular structure with more than one type of linking moieties.

Thus, according to an aspect of some embodiments of the present invention, there is provided a process of preparing the molecular structure presented herein, which includes linking a first functional group on a HA moiety (any of Groups A-D, or other functionalities if HA is pre-modified to exhibit a modified reactive group) to a first functional group on CBD (any of Groups E-F, or other functionalities if CBD is pre-modified to exhibit a modified reactive group) via (forming) a first biocleavable linking moiety.

The above process may further include, linking CBD to a different functional group on a HA moiety, thereby loading more CBD via a second biocleavable linking moiety. The process may include additional loading steps to load more CBD via a third biocleavable linking moiety, and so on.

As discussed hereinabove, in some embodiments, the process starts with an optional step of modifying a native functional group on the HA strand such that the HA exhibits a modified functional group. In such embodiments, modification of functional groups in the HA strand facilitates the synthesis and the sequential attachment of CBD moieties. Thereafter, the process continues as presented hereinabove.

According to some embodiments, some or all the steps of linking the various components of the molecular structure to one another further includes attaching controlled and sequential removal a variety of protection groups on the various functional groups. It is noted herein that similar protecting groups can be used to render one or more of the bioactive agents prodrugs/precursors of the same, once the protecting group is cleaved-off the bioactive agent; this practice is particularly useful in cases of bioactive agents that have more than two reactive functional groups, which need to be protected during the string elongation process.

As used herein, the term “protecting group” or “suitable protecting group”, refers to amino protecting groups, hydroxyl protecting groups and the like, depending on its location within the compound and includes those described in detail in Protecting Groups in Organic Synthesis, T. W. Greene and P. G. M. Wuts, 3rd edition, John Wiley & Sons, 1999.

It is expected that during the life of a patent maturing from this application many relevant molecular structures will be developed and the scope of the phrase “molecular structure” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Example 1

### Synthesis of Exemplary Molecular Structures

Following is an exemplary synthesis that demonstrates the preparation of an exemplary molecular structure, according to some embodiments of the present invention.

In this example, CBD is coupled directly to HA via an biocleavable ester linking moiety, thereby forming an exemplary molecular structure according to some embodiments of the present invention. It is noted that although the language refers to a single CBD moiety, multiple CBD moieties are attached simultaneously on multiple sites along the HA strand.

Scheme 3 presents an exemplary process for obtaining an exemplary molecular structure, according to some embodiments of the present invention, comprising a hyaluronic acid residue, and a plurality of CBD residues attached thereto, wherein only a single CBD residue is depicted to show the biocleavable moiety (ester) which is formed by reacting a hydroxyl (Group E) in CBD with a carboxyl on the HA (Group A).

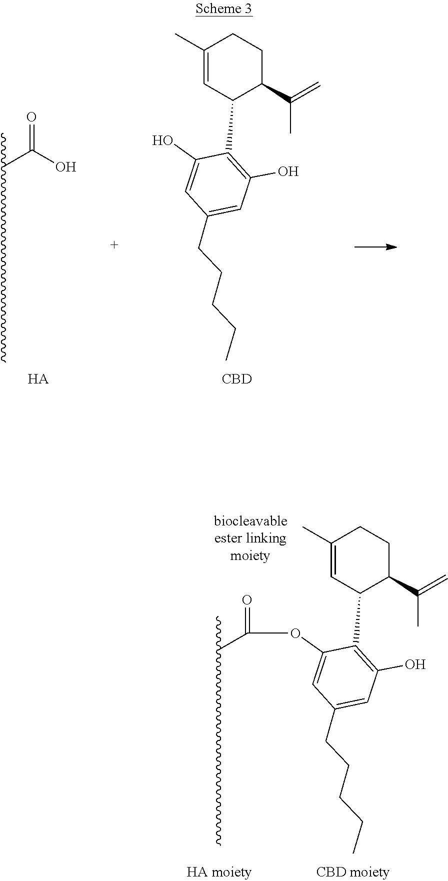

Scheme 4 presents an exemplary process for obtaining an exemplary molecular structure, according to some embodiments of the present invention, comprising a step of modifying a hydroxyl functionality on a CBD molecule, using β-alanine as part of a biocleavable linking moiety, in preparation for attachment to a carboxyl functionality on the hyaluronic acid strand.

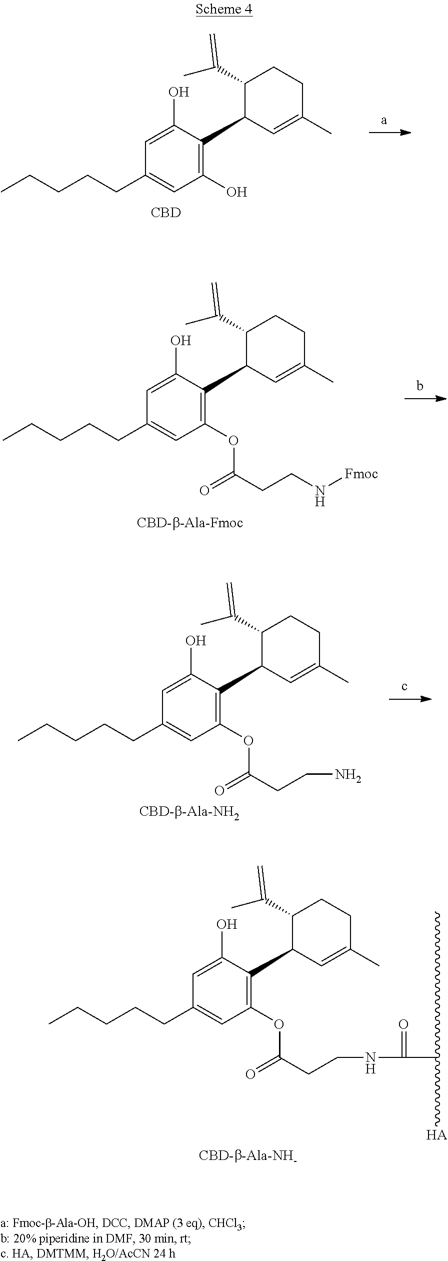

### Example 2

### Activity Assays

The molecular structures provided herein are tested for efficacy and safety according to an exemplary experimental protocol as follows.

Administration is effected by 0.2 mL subdermal injections using 30G or 27G hypodermic needles.

The dose frequency is 6 injection sites on the back of a rat administered once.

In-life study duration/handling is 13 weeks/14 weeks.

Morbidity and mortality: Twice a day.

Detailed clinical observation: prior to dosing, frequently for the first two hours post dosing, and twice weekly thereafter.

Grading for erythema and edema daily for the first 14 days, or until disappearance of erythema and edema (see table below).

Injection site measurement by caliper starting a day after injection and twice weekly thereafter.

A photograph of the back of each animal, right after injection and at 1, 2 and 3 months (before termination) after injection. A hair removal cream will be used before each photograph session.

Body weight monitoring: during acclimation and twice a week thereafter.

Necropsy and gross pathology: macroscopic findings on all main study animals at termination with special attention to draining lymph nodes.

Following termination, each animal receives one subdermal injection of 0.2 mL of a control or test article. The injected site is immediately sampled and fixed. These samples will allow the structural observation and histopathologic comparison with the injection sites sampled 13 weeks after injection.

Organ weight monitoring: any tissue with abnormal findings.

Tissue preservation: injection sites and any tissue with abnormal findings.

Histology/Pathology: Injection site will stained with, masson trichrome, hematoxylin eosin, and alcian blue (5 slides).

Grading system for histology: The qualitative and semi-quantitative evaluation of the histologic slides is conducted according to the ISO 10993.

For each test and control product, the mean irritation score is calculated among the 4 injection sites, each day.

Grading system for histologic evaluation (cell type/Response): polymorphonuclear cells, lymphocytes, plasma cells, macrophages, giant cells, necrosis, neovascularization, fibrosis, fatty infiltrate, fibrin, hemorrhage, fibroplasia, tissue integration, tissue ingrowth, encapsulation, product degradation.

Irritant Ranking Score (IRS, Table, Determination of IRS; ISO 10993): the individual irritation scores of the test and control products is calculated based on the histologic gradings as the sum F.1 of the tissue damage and cellular inflammatory parameter scores weighted with a factor 2 plus the sum F.2 of the repair phase of inflammation and fatty infiltrate parameter scores. The average individual irritation score (group average) is calculated as the mean result of the 3 injection sites per tested product. The IRS is determined by subtracting the control product (C1) average score to the test product average score. The IRS calculation is rounded to the nearest 0.1. A negative difference is recorded as zero.

The IRS is graded as follows:
non-irritant (0.0 to 2.9)
slight irritant (3.0 to 8.9)
moderate irritant (9.0 to 15.0)
severe irritant (= or >15.1).

TABLE 1

| | Product (T1, T2, T3, C1 or C2) | | |
|---|---|---|---|
| | Site 1 | Site 2 | Site 3 |
| F.1 Inflammation | | | |
| Polymorphonuclear cells | | | |
| Lymphocytes | | | |
| Plasma cells | | | |
| Macrophages | | | |
| Giant cells | | | |
| Necrosis | | | |
| F.1 SUBTOTAL ×2 | | | |
| F.2 Neovascularization | | | |
| Neovascularization | | | |
| Fibrosis | | | |
| Fatty infiltrate | | | |
| F.2 SUB TOTAL | | | |
| TOTAL F.1 + F.2 | | | |
| GROUP AVERAGE | | | |

| | |
|---|---|
| IRS vs control C1 | IRS = Group average − Group average for C1 |
| IRS vs control C2 | IRS = Group average − Group average for C2 |

### Example 3

### Safety Studies

Pigmentary disorders in dermatology, such as lentigines, post-inflammatory hyperpigmentation and melasma, are not adequately treated at the present time. Lentigines or age spots are of universal occurrence in Caucasians due to the general aging process and exposure to sunlight. Melasma, which is characterized by blotchy, brown hyperpigmentation of the face, occurs in a high percentage of women on oral contraceptives. Post-inflammatory hyperpigmentation can accompany many skin diseases including chronic eczema, lichen planus and psoriasis. While some of these lesions can be treated with cryotherapy, alternative pharmacological approaches would be more readily accepted by both physicians and patients.

Several active agents are used to treat melisma, and include hydroquinone, tanexamic acid, kojic acid, cysteamine, azelaic acid, arbutin and the likes. These agents require long term use to produce depigmentation also because limited biostability in dermis.

The HA-CBD conjugates, according to some embodiments of the present invention, allow “slow” or controlled release of CBD, as well as improved modalities, increase half-life, and improved efficacy in treatment of melasma.

The safety of the herein disclosed HA-CBD conjugates was tested as follows. One male brown pig age 6 months was housed in standard stainless steel pen at the Animal Facilities, Havat Keshet, Rehovot, and acclimated for at least two weeks prior to initiation of the study.

Prior to treatment, melanocytes were stimulated by UV irradiation following a published protocol [Nair, X. et al., Journal of Investigative Dermatology, 1993, 101 (2), pp. 145-149]. A pattern of square areas, each 4 cm×4 cm in size and spaced 2 cm apart were chosen on the side of the animal for the treatment. Thereafter, the tested samples were dissolved in water/glycerol (2:1), and applied on the squares using a microneedling (mesotherapy) device at 2.5 mm depth. The treatment was delivered once a month for 3 months, affording three treatments in total.

The tested samples were:
1. HA-(PEG)₃-CBD (20 wt. %)

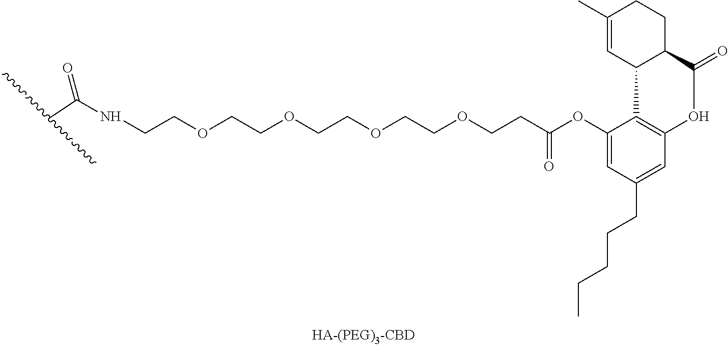

2. HA-(AiB)-CBD (10 wt. %)

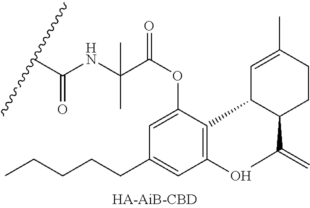

3. HA-(βAla)-CBD (10 wt. %)

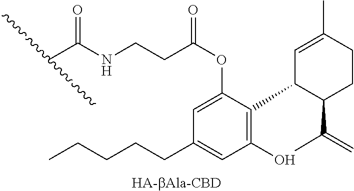

4. CBD+HA mix as a reference (2.8 mg of CBD. 17.2 mg of HANa)
5. Untreated

At the end of experiment biopsies were taken and subjected to histopathology according to published protocols [Kerlin, R. et al., Toxicol Pathol., 2016, 44 (2), pp. 147-62; Schafer, K. A. et al., Toxicol Pathol., 2018, 46 (3), pp. 256-265].

### Organ/Tissue Collection and Fixation:

Samples of skin (n=5) from one pig were harvested, fixed in 4% formaldehyde, and kept in the fixative for 48 hours for further fixation. The tissues were trimmed, put in embedding cassettes and processed routinely for paraffin embedding.

### Slide Preparation:

Paraffin sections (4 microns thick) were cut, put on glass slides and stained with Hematoxylin & Eosin (H&E) for general histopathology, Masson Trichrome (MT) for collagen and Masson Fontana (MF) for melanin.

### Light Microscopy Photography:

Pictures were taken, using Olympus microscope (BX60, serial NO. 7D04032) equipped with microscope's Camera (Olympus DP73, serial NO. OH05504) at objective magnification of X10.

### Histopathological Evaluation:

The H&E-stained slides were examined, described and scored by the study Pathologist, using a semi-quantitative grading scale, of 5-point scale, for the severity of the histopathological changes as follows:
Grade 0—The tissue appears normal.
Grade 1—Minimal pathological findings.
Grade 2—Mild pathological findings.
Grade 3—Moderate pathological findings.
Grade 4—Severe pathological findings.

The Masson Trichrome (MT) stained slides were examined and graded by a semi-quantitative scoring system for the presence of fibrosis/collagen (mag. X10), as follows:
0—No signs of fibrosis/collagen degradation as appears in normal skin.
1—Mild fibrosis/collagen degradation.
2—Moderate fibrosis/collagen degradation.
3—Severe fibrosis/collagen degradation.

The Masson Fontana (MF) stained slides were examined and graded by a semi-quantitative scoring system for the presence of melanin (mag. X10), as follows:
0—Almost no pigmented cells visible;
1—Few pigmented cells, less than in normal skin;
2—Normal number of pigmented cells, as compared to normal skin;
3—Increased number of pigmented cells;
4—High number of pigmented cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating a skin condition in a subject in need thereof, comprising administering to the subject an effective amount of a molecular structure that comprises a hyaluronic acid (HA) moiety and a plurality of cannabidiol (CBD) moieties attached thereto via a biocleavable linking moiety, wherein the skin condition is selected from the group consisting of melasma, skin whitening, hyperpigmentation, Chadwick's sign, Linea alba, Perineal raphe, acne scarring, acne, liver spots, surgical scars, stretch marks and hair loss.

2. The method of claim 1, wherein said administering is effected topically, subcutaneous, and/or percutaneously.

3. The method of claim 2, wherein said administering is effected by a percutaneous and/or a minimally invasive tool.

4. The method of claim 3, wherein said administering is effected by microneedling.

5. The method of claim 1, wherein said biocleavable linking moiety is selected from the group consisting of amide, ester, carbonate, carbamate, thiocarbamate, sulfonamide, and phosphate.

6. The method of claim 5, wherein said biocleavable linking comprises a moiety selected from the group consisting of a Gly moiety, a B-Ala moiety, a GABA moiety, a 3-Amino-2 moiety, a 2-dimethyl-propionic acid moiety, a sarcosine moiety, and a NH₂-PEG4-propionic acid moiety.

7. The method of claim 5, wherein said cannabidiol moieties are attached to said hyaluronic acid moiety via said biocleavable linking moiety and further via a spacer moiety.

8. A cosmetic composition for treating a skin condition, comprising a hyaluronic acid (HA) moiety and a plurality of cannabidiol (CBD) moieties attached thereto via a biocleavable linking moiety, and a cosmetically acceptable carrier, wherein the skin condition is selected from the group consisting of melasma, skin whitening, hyperpigmentation, Chadwick's sign, Linea alba, Perineal raphe, acne scarring, acne, liver spots, surgical scars, stretch marks and hair loss.
